(19) United States
(12) Patent Application Publication
(10) Pub. No.: US 2010/0056595 A1
(43) Pub. Date: Mar. 4, 2010

(54) Pyrazole Derivatives as P2X7 Modulators

(75) Inventors: Paul John Beswick, Essex (GB); David Kenneth Dean, Essex (GB); Daryl Simon Walter, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited, Greenford, EN (GB)

(21) Appl. No.: 12/595,099

(22) PCT Filed: Apr. 10, 2008
(86) PCT No.: PCT/EP08/54382

§ 371 Date: Oct. 8, 2009

(30) Foreign Application Priority Data
Apr. 11, 2007 (GB) 0707009.7
Mar. 31, 2008 (GB) 08505815.8

(51) Int. Cl. A61K 31/415; C07D 231/12; A61P 25/00

(52) U.S. Cl. 514/406; 548/376.1

(57) ABSTRACT

The present invention relates to a compound of formula (I) or a pharmaceutically acceptable salt thereof:

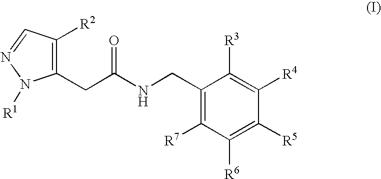

wherein R¹ represents C₁₋₆ alkyl or C₃₋₆ cycloalkyl, either of which is optionally substituted with 1, 2 or 3 halogen atoms; and
R² represents hydrogen, halogen, C₁₋₆ alkyl or C₃₋₆ cycloalkyl; and either of said C₁₋₆ alkyl or C₃₋₆ cycloalkyl is optionally substituted with 1, 2 or 3 halogen atoms.

The pyrazole compounds of formula (I) or salts thereof modulate P2X7 receptor function and are capable of antagonizing the effects of ATP at the P2X7 receptor (P2X7 receptor antagonists). The invention also relates to the use of such compounds or salts, or pharmaceutical compositions thereof, in the treatment or prevention of disorders/diseases mediated by the P2X7 receptor, for example pain, inflammation or a neurodegenerative disease.

The present invention relates to pyrazole derivatives which modulate P2X7 receptor function and are capable of antagonizing the effects of ATP at the P2X7 receptor (“P2X7 receptor antagonists”); to processes for their preparation; pharmaceutical compositions containing them; and to the use of such compounds in therapy.

The P2X7 receptor is a ligand-gated ion-channel which is expressed in cells of the hematopoietic lineage, e.g. macrophages, microglia, mast cells, and lymphocytes (T and B) (see, for example, Collo, et al. Neuropharmacology, Vol. 36, pp 1277-1283 (1997)), and is activated by extracellular nucleotides, particularly adenosine triphosphate (ATP). Activation of P2X7 receptors has been implicated in giant cell formation, degranulation, cytolytic cell death, CD62L shedding, regulation of cell proliferation, and release of proinflammatory cytokines such as interleukin 1 (IL-1β) and tumour necrosis factor (TNFα) (e.g. Hide, et al. Journal of Neurochemistry, Vol 75, pp 965-972 (2000)). P2X7 receptors are also located on antigen presenting cells, keratinocytes, parotid cells, hepatocytes, erythrocytes, erythroleukaemic cells, monocytes, fibroblasts, bone marrow cells, neurones, and renal mesangial cells. Furthermore, the P2X7 receptor is expressed by presynaptic terminals in the central and peripheral nervous systems and has been shown to mediate glutamate release in glial cells (Anderson, C. et al. Drug. Dev. Res., Vol. 50, page 92 (2000)).

The localisation of the P2X7 receptor to key cells of the immune system, coupled with its ability to release important inflammatory mediators from these cells suggests a potential role of P2X7 receptor antagonists in the treatment of a wide range of diseases including pain and neurodegenerative disorders. Recent preclinical in vivo studies have directly implicated the P2X7 receptor in both inflammatory and neuropathic pain (Dell'Antonio et al., Neurosci. Lett., 327, pp 87-90, 2002, Chessell, I P., et al., Pain, 114, pp 386-396, 2005) while there is in vitro evidence that P2X7 receptors mediate microglial cell induced death of cortical neurons (Skaper, S. D., et al., Program No. 937.7. 2005 Abstract Viewer/Itinerary Planner. Washington, D.C.: Society for Neuroscience, 2005. Online). In addition, up-regulation of the P2X7 receptor has been observed around β-amyloid plaques in a mouse model of Alzheimer's disease (Parvathenani, L. et al. J. Biol. Chem., Vol. 278(15), pp 13309-13317, 2003).

The present invention provides compounds which modulate P2X7 receptor function and are capable of antagonizing the effects of ATP at the P2X7 receptor (“P2X7 receptor antagonists”).

A first aspect of the present invention provides a compound of formula (I) or a pharmaceutically acceptable salt thereof:

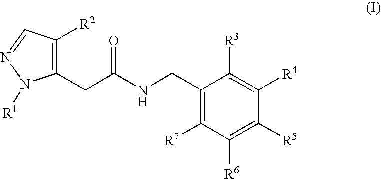

wherein:

R¹ represents C₁₋₆ alkyl or C₃₋₆ cycloalkyl, either of which is optionally substituted with 1, 2 or 3 halogen (e.g. fluorine) atoms;

R² represents hydrogen, halogen, C₁₋₆ alkyl or C₃₋₆ cycloalkyl; and either of said C₁₋₆ alkyl or C₃₋₆ cycloalkyl is optionally substituted with 1, 2 or 3 halogen (e.g. fluorine) atoms; and

R³, R⁴, R⁵, R⁶ and R⁷ independently represent hydrogen, halogen (e.g. fluorine or chlorine), cyano, C₁₋₆ alkyl (e.g. methyl), C₂₋₆ alkenyl, C₂₋₆ alkynyl, C₃₋₆ cycloalkyl or phenyl, and any of said C₁₋₆ alkyl, C₂₋₆ alkenyl, C₂₋₆ alkynyl, C₃₋₆ cycloalkyl or phenyl is optionally substituted with 1, 2 or 3 halogen (e.g. fluorine) atoms; or R⁶ and R⁷ together with the carbon atoms to which they are attached form a benzene ring which is optionally substituted with 1, 2 or 3 halogen atoms;

with the proviso that when R³ and R⁷ independently represent hydrogen or fluorine, at least one (e.g. one or two, e.g. one) of R⁴, R⁵ and R⁶ is a halogen atom, or only one of R⁴, R⁵ and R⁶ is a CF₃ group.

As used herein, the term “alkyl” (when used as a group or as part of a group) refers to a straight or branched hydrocarbon chain containing the specified number of carbon atoms. For example, C₁₋₆ alkyl means a straight or branched hydrocarbon chain containing at least 1 and at most 6 carbon atoms. Examples of alkyl include, but are not limited to; methyl (Me), ethyl (Et), n-propyl, i-propyl, n-hexyl and i-hexyl.

As used herein, the term “alkenyl” refers to a straight or branched hydrocarbon chain containing the specified number of carbon atoms wherein at least one (e.g. one) carbon-carbon bond is a double bond. Examples of alkenyl include, but are not limited to ethenyl, propenyl, n-butenyl, i-butenyl, n-pentenyl and i-pentenyl.

As used herein, the term “alkynyl” refers to a straight or branched hydrocarbon chain containing the specified number of carbon atoms wherein at least one (e.g. one) carbon-carbon bond is a triple bond. Examples of alkynyl include, but are not limited to ethynyl, propynyl, butynyl, i-pentynyl, n-pentynyl, i-hexynyl and n-hexynyl.

The term ‘cycloalkyl’ unless otherwise stated (e.g. by virtue of a different specified number of carbon atoms) means a closed 3 to 8 membered non-aromatic ring, for example cyclopropyl, cyclobutyl, cyclopentyl, cyclohexyl, cycloheptyl or cyclooctyl.

The term ‘halogen’ is used herein to describe, unless otherwise stated, a group selected from fluorine, chlorine, bromine and iodine.

It is to be understood that the present invention covers and discloses all possible combinations of particular, preferred, suitable, or other embodiments of groups or features (e.g. of R¹, R², R³, R⁴, R⁵, R⁶ and/or R⁷), e.g. all possible combinations of embodiments of different groups or features, which embodiments are described herein.

In certain embodiments of the invention, R¹ represents C₁₋₆ alkyl (e.g. methyl, ethyl, n-propyl or i-propyl) optionally substituted with 1, 2 or 3 halogen (e.g. fluorine) atoms. In one embodiment, R¹ represents C₁₋₄ alkyl (e.g. methyl), trifluoromethyl or trifluoroethyl (e.g. —CH₂—CF₃).

In one particular embodiment, R¹ represents methyl or trifluoroethyl, such as methyl or —CH₂—CF₃.

In one particular embodiment, R¹ represents methyl, ethyl, or ethyl substituted with 1, 2 or 3 fluorine atoms (e.g. trifluoroethyl such as —CH₂—CF₃).

Preferably, R¹ represents methyl.

In certain embodiments of the invention, R² represents hydrogen, unsubstituted C₁₋₆ alkyl or halogen.

In a particular embodiment, R² represents hydrogen, methyl or halogen.

Preferably, R² represents methyl or halogen; for example methyl, fluorine, chlorine or bromine.

In one embodiment of the invention, R³, R⁴, R⁵, R⁶ and R⁷ independently represent hydrogen, halogen, cyano, trifluoromethyl or unsubstituted C₁₋₆ alkyl.

In a particular embodiment, R³, R⁴, R⁵, R⁶ and R⁷ independently represent hydrogen, halogen, cyano, methyl or trifluoromethyl. In a more particular embodiment, R³, R⁴, R⁵, R⁶ and R⁷ independently represent hydrogen, chlorine, fluorine, bromine, methyl or trifluoromethyl.

Preferably,

R³ represents chlorine or methyl (in particular chlorine), R⁴ represents trifluoromethyl, and R⁵, R⁶ and R⁷each represent hydrogen;

R³ represents chlorine, R⁵ represents fluorine, and R⁴, R⁶ and R⁷ each represent hydrogen;

R³represents chlorine, R⁴ and R⁵ both represent fluorine, and R⁶ and R⁷ both represent hydrogen; or

R³and R⁵ both represent chlorine, and R⁴, R⁶and R⁷each represent hydrogen.

In an alternative particular embodiment, R³ and R⁵ both represent chlorine, R⁴ and R⁶ both represent hydrogen, and R⁷ represents methyl, fluorine or chlorine.

In all embodiments of the invention herein described, when R³ and R⁷ independently represent hydrogen or fluorine, at least one (e.g. one or two, e.g. one) of R⁴, R⁵ and R⁶ is a halogen atom, or only one of R⁴, R⁵ and R⁶ is a CF₃ group.

In a particular embodiment of the invention herein described, when R³ and R⁷ independently represent hydrogen or fluorine, at least one (e.g. one or two, e.g. one) of R⁴, R⁵ and R⁶ is a halogen atom.

In a particular embodiment of the invention, there is provided a compound of formula (I), or a pharmaceutically acceptable salt thereof, wherein:

R¹ represents C₁₋₆ alkyl optionally substituted with 1, 2 or 3 halogen (e.g. fluorine) atoms (in particular, wherein R¹ represents methyl or trifluoroethyl);

R² represents hydrogen, methyl or halogen; and

R³, R⁴, R⁵, R⁶ and R⁷ independently represent hydrogen, chlorine, fluorine, bromine, methyl or trifluoromethyl;

with the proviso that when R³ and R⁷ independently represent hydrogen or fluorine, at least one (e.g. one or two, e.g. one) of R⁴, R⁵ and R⁶ is a halogen atom, or only one of R⁴, R⁵ and R⁶ is a CF₃ group (in particular, wherein when R³ and R⁷ independently represent hydrogen or fluorine, at least one of R⁴, R⁵ and R⁶ is a halogen atom).

A particular aspect of the invention provides a compound selected from examples E1 to E8, as shown and/or named below, or a pharmaceutically acceptable salt thereof.

Another particular aspect of the invention provides a compound selected from examples E9 to E20, as shown and/or named below, or a pharmaceutically acceptable salt thereof.

Antagonists of P2X7 may be useful in preventing, treating, or ameliorating a variety of pain states (e.g. neuropathic pain, chronic inflammatory pain, and visceral pain), inflammation and neurodegeneration, in particular Alzheimer's disease. P2X7 antagonists may also constitute useful therapeutic agents in the management of rheumatoid arthritis and inflammatory bowel disease.

Compounds of the present invention which modulate P2X7 receptor function and are capable of antagonizing the effects of ATP at the P2X7 receptor (“P2X7 receptor antagonists”) may be competitive antagonists, inverse agonists, or negative allosteric modulators of P2X7 receptor function.

Certain compounds of formula (I) may in some circumstances form acid addition salts thereof. It will be appreciated that for use in medicine compounds of formula (I) may be used as salts, in which case the salts should be pharmaceutically acceptable. Pharmaceutically acceptable salts include those described by Berge, Bighley and Monkhouse, J. Pharm. Sci., 1977, 66, 1-19. When a compound of the present invention is basic, in one embodiment a pharmaceutically acceptable salt is prepared from a pharmaceutically acceptable acid, such as an inorganic or organic acid. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In a particular embodiment, the pharmaceutically acceptable acid is benzenesulfonic, camphorsulfonic, ethanesulfonic, hydrobromic, hydrochloric, methanesulfonic, nitric, phosphoric, sulfuric, or p-toluenesulfonic acid.

Examples of pharmaceutically acceptable salts include salts formed from maleic, fumaric, benzoic, ascorbic, pamoic, succinic, hydrochloric, sulfuric, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

The compounds of formula (I) or salts thereof may be prepared in crystalline or non-crystalline form, and, if crystalline, may optionally be solvated, e.g. as the hydrate. This invention includes within its scope solvates (e.g. hydrates), such as stoichiometric solvates (e.g. hydrates), of the compounds or salts thereof, as well as compounds or salts thereof containing variable amounts of solvent (e.g. water).

Certain compounds of formula (I) or salts thereof may be capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The subject invention also includes isotopically-labeled compounds, which are identical to those recited in formula (I), or salts thereof, but for the fact that one or more atoms are replaced by an atom having an atomic mass or mass number different from the atomic mass or mass number most commonly found in nature. Examples of isotopes that can be incorporated into compounds or salts of the invention include isotopes of hydrogen, carbon, nitrogen, oxygen, phosphorous, fluorine, iodine, and chlorine, such as 3H, 11C, 14C, 18F, 123I and 125I.

Compounds of the present invention and pharmaceutically acceptable salts of said compounds that contain the aforementioned isotopes and/or other isotopes of other atoms are within the scope of the present invention. Isotopically-labeled compounds or salts of the present invention, for example those into which radioactive isotopes such as 3H, 14C are incorporated, are potentially useful in drug and/or substrate tissue distribution assays. Tritiated, i.e., 3H, and carbon-14, i.e., 14C, isotopes are for example optionally chosen for their ease of preparation and detectability. 11C and 8F isotopes are generally useful in PET (positron emission tomography), and 125I isotopes are generally useful in SPECT (single photon emission computerized tomography). PET and SPECT are useful in brain imaging. Further, substitution with heavier isotopes such as deuterium, i.e., 2H, can sometimes afford certain effects resulting from greater metabolic stability, for example increased in vivo half-life or reduced dosage requirements and, hence, may be chosen in some circumstances. Isotopically labeled compounds of formula (I) or salts thereof of this invention are in one embodiment and in some cases prepared by carrying out the procedures disclosed in the Schemes and/or in the Examples below, by substituting a readily available isotopically labeled reagent for a non-isotopically labeled reagent.

A further particular aspect of the invention provides a compound of formula (I) or a pharmaceutically acceptable salt thereof which is not a radioactive isotopically labeled compound or salt. In a particular embodiment, the compound or salt is not an isotopically labeled compound or salt.

Preparation of Compounds

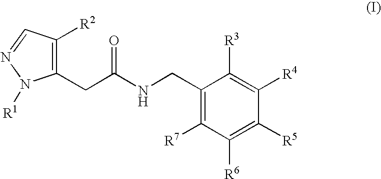

Compounds of formula (I), wherein the variables are defined above, and salts and solvates thereof may be prepared by the methodology described hereinafter, constituting a further aspect of this invention.

According to a further aspect of the invention, there is provided a process for preparing a compound of formula (I) or a pharmaceutically acceptable salt thereof which comprises:

(a) Coupling of a carboxylic acid of formula (2) (or an activated derivative thereof) with an amine of formula (3) (see Scheme 1), wherein R¹, R², R³, R⁴, R⁵, R⁶, and R⁷ are as defined above. Compounds (2) and (3) are optionally protected.

(b) Deprotecting a compound of formula (I) which is protected. Examples of protecting groups and the means for their removal can be found in T.W. Greene and P.G.M. Wuts ‘Protective Groups in Organic Synthesis’ (J.Wiley and Sons, 3^{rd} Ed. 1999).

(c) Interconversion of a compound of formula (I) to another compound of formula (I). Examples of interconversion procedures include halogenation, oxidation, reduction, alkylation, aromatic substitution, nucleophilic substitution, amide coupling and ester hydrolysis. In a particular embodiment, the interconversion comprises halogenation (e.g. fluorination, chlorination, bromination, or iodination) of a suitable compound of formula (I); see Scheme 4 and surrounding text below for more details.

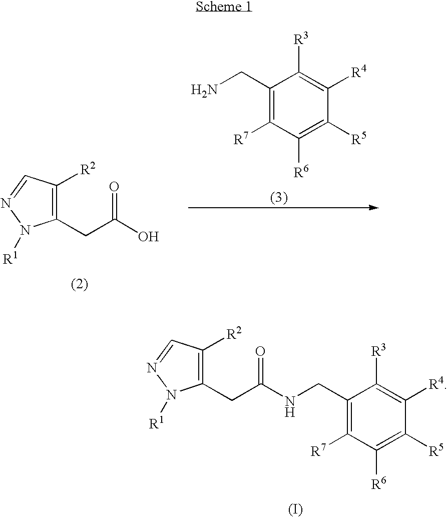

The coupling of an acid of formula (2) and an amine of formula (3) typically comprises the use of an activating agent or agents, such as water soluble carbodiimide (such as 1-ethyl-3-(3-dimethylaminopropyl)carbodiimide hydrochloride) or polymer-supported carbodiimide, 1-hydroxybenzotriazole (HOBT) and/or 1-Hydroxy-7-azabenzotriazole (HOAt), and optionally a suitable base such as a tertiary alkylamine (e.g. diisopropylethylamine, N-ethyl morpholine, triethylamine) or pyridine, in a suitable solvent such as DMF and/or dichloromethane and e.g. at a suitable temperature e.g. between 0° C. and room temperature. Alternatively the coupling of (2) and (3) may be accomplished by treatment with O-(7-Azabenzotriazol-1-yl)-N,N,N′,N′-tetramethyluronium hexafluorophosphate and a suitable tertiary alkylamine such as diisopropylethylamine in a suitable solvent such as dimethylformamide e.g. at a suitable temperature such as room temperature. Where the compound of formula (2) is an activated derivative (e.g. acid chloride, mixed anhydride, active ester (e.g. O-acyl-isourea)), process (a) typically comprises treatment of said activated derivative with an amine (Ogliaruso, M. A.; Wolfe, J. F. in The Chemistry of Functional Groups (Ed. Patai, S.) Suppl. B: The Chemistry of Acid Derivatives, Pt. 1 (John Wiley and Sons, 1979), pp 442-8; Beckwith, A. L. J. in The Chemistry of Functional Groups (Ed. Patai, S.) Suppl. B: The Chemistry of Amides (Ed. Zabricky, J.) (John Wiley and Sons, 1970), pp 73 ff.

Representative methods for the preparation of compounds of formula (2) are shown in Schemes 2-3 below:

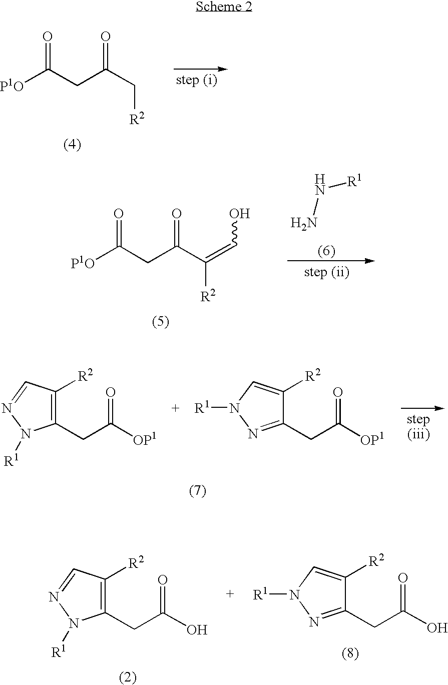

Wherein R¹ and R² are as defined above and P¹ represents a suitable protecting group such as C₁₋₆ alkyl (e.g. methyl).

Step (i) typically comprises treatment of (4) with suitable base(s) such as sodium hydride and n-butyl lithium and a suitable formylating agent such as ethyl formate in a suitable solvent (e.g. dry solvent) such as tetrahydrofuran at a suitable temperature e.g. between −20° C. and room temperature.

Step (ii) typically comprises the use of a hydrazine of formula (6) or a salt thereof (e.g. HCl) in a suitable solvent such as tetrahydrofuran or water at a suitable temperature such as room temperature.

Step (iii) typically comprises a standard procedure for conversion of a carboxylic ester to an acid, such as use of an appropriate hydroxide salt (e.g. sodium hydroxide, e.g. aqueous, e.g. 2N aqueous) in an appropriate solvent such as a methanol at a suitable temperature such as between room temperature and about 70° C.

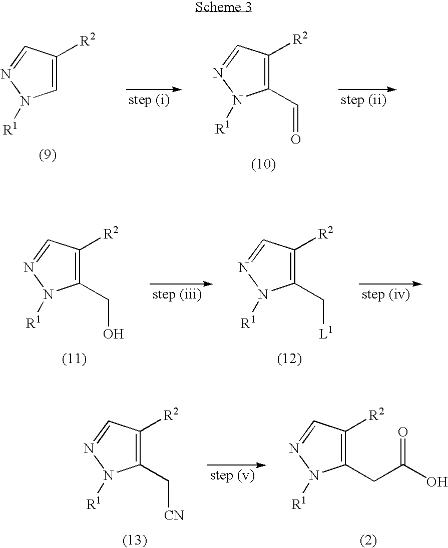

Wherein R¹ and R² are as defined above and L¹ represents a suitable leaving group such as halogen (e.g. chlorine or bromine).

Analagous processes have been described previously in the chemical literature (see J. Heterocyclic Chem., 30, 997 (1993), Bioorganic and Medicinal Chemistry, 9, 961 (2001)).

Step (i) typically comprises treatment of (9) with a suitable alkyllithium reagent such as n-butyl lithium and a suitable formylating agent such as dimethylformamide in a suitable solvent such as tetrahydrofuran at a suitable temperature e.g. between −70° C. and room temperature.

Step (ii) typically comprises the use of a suitable reducing agent such as sodium borohydride in a suitable solvent such as ethanol at a suitable temperature such as between 5° C. and room temperature.

Step (iii) typically comprises a standard procedure for conversion of an alcohol into a leaving group, such as the use of thionyl chloride (for when L¹ represents chlorine) in an appropriate solvent such as dichloromethane at a suitable temperature such as room temperature.

Step (iv) typically comprises treatment of (12) with a cyanide salt such as sodium cyanide in a suitable solvent such as dimethylsulphoxide at a suitable temperature such as between room temperature and 50° C.

Step (v) typically comprises a method for converting a nitrile into a carboxylic acid such as by treatment with a suitable acid such as 5M aqueous hydrochloric acid in a suitable solvent such as 1,4-dioxane and at a suitable temperature such as between room temperature and 100° C.

Compounds of the general formulae (3), (4), (6) and (9) are typically either available from commercial sources or can be prepared by a person skilled in the art e.g. using methods described in the chemical literature (or using analogous methods).

In an alternative embodiment, process route (c), involving interconversion of a compound of formula (I) to another compound of formula (I), can comprise halogenation (e.g. fluorination, chlorination, bromination, or iodination) of a suitable compound of formula (I), for example halogenation of a compound of formula (I) wherein R² represents H to prepare a compound of formula (I) wherein R² represents halogen (e.g. fluorine, chlorine, bromine or iodine), as shown in Scheme 4 below.

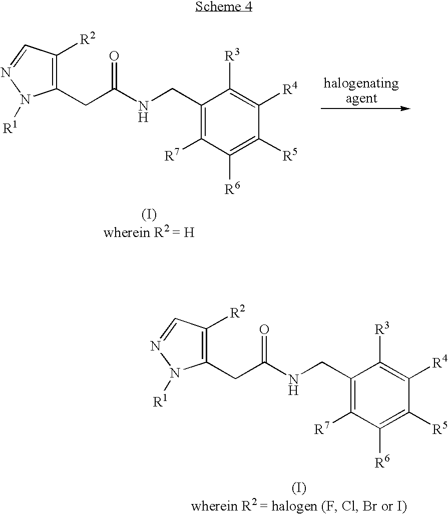

Chlorination or bromination or iodination is for example carried out using a chlorinating agent such as N-chlorosuccinimide or a brominating agent such as N-bromosuccinimide or a iodinating agent such as N-iodosuccinimide, in a suitable solvent such as N,N-dimethylformamide, e.g. at a suitable temperature such as from room temperature to about 60° C. Fluorination is for example carried out using a fluorinating agent such as 1-(chloromethyl)-4-fluoro-1,4-diazoniabicyclo[2.2.2]octane ditetrafluoroborate (CAS[140681-55-6], e.g. Selectfluor™, available e.g. from Aldrich), in a suitable solvent such as N,N-dimethylformamide, e.g. at a suitable temperature such as about 50° C.

Pharmaceutically acceptable salts may for example be prepared conventionally by reaction (mixture) with the appropriate acid or acid derivative.

Clinical Indications

It is believed that, as compounds or pharmaceutically acceptable salts of the present invention modulate P2X7 receptor function and are capable of antagonizing the effects of ATP at the P2X7 receptor (P2X7 receptor antagonists), they may be useful in the treatment of pain, including acute pain, chronic pain, chronic articular pain, musculoskeletal pain, neuropathic pain, inflammatory pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, cancer chemotherapy, headache, toothache and dysmenorrhea.

Chronic articular pain conditions include rheumatoid arthritis, osteoarthritis, rheumatoid spondylitis, gouty arthritis and juvenile arthritis.

Pain associated with functional bowel disorders includes non-ulcer dyspepsia, non-cardiac chest pain and irritable bowel syndrome.

Neuropathic pain syndromes include: diabetic neuropathy, sciatica, non-specific lower back pain, trigeminal neuralgia, multiple sclerosis pain, fibromyalgia, HIV-related neuropathy, post-herpetic neuralgia, trigeminal neuralgia, and pain resulting from physical trauma, amputation, phantom limb syndrome, spinal surgery, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Other conditions which could potentially be treated by compounds or pharmaceutically acceptable salts of the present invention include fever, inflammation, immunological diseases, abnormal platelet function diseases (e.g. occlusive vascular diseases), impotence or erectile dysfunction; bone disease characterised by abnormal bone metabolism or resorbtion; hemodynamic side effects of non-steroidal anti-inflammatory drugs (NSAID's) and cyclooxygenase-2 (COX-2) inhibitors, cardiovascular diseases; neurodegenerative diseases and/or neurodegeneration, neurodegeneration following trauma, tinnitus, dependence on a dependence-inducing agent such as opiods (e.g. morphine), CNS depressants (e.g. ethanol), psychostimulants (e.g. cocaine) and nicotine; complications of Type I diabetes, kidney dysfunction, liver dysfunction (e.g. hepatitis, cirrhosis), gastrointestinal dysfunction (e.g. diarrhoea), colon cancer, overactive bladder and urge incontinence. Depression and alcoholism could potentially also be treated by compounds or pharmaceutically acceptable salts of the present invention.

Inflammatory conditions include skin conditions (e.g. sunburn, burns, eczema, dermatitis, allergic dermatitis, psoriasis), meningitis, ophthalmic diseases such as glaucoma, retinitis, retinopathies, uveitis and of acute injury to the eye tissue (e.g. conjunctivitis), inflammatory lung disorders (e.g. asthma, bronchitis, emphysema, allergic rhinitis, respiratory distress syndrome, pigeon fancier's disease, farmer's lung, chronic obstructive pulmonary disease (COPD), airways hyperresponsiveness); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation and other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome.

Immunological diseases include autoimmune diseases, immunological deficiency diseases or organ transplantation.

Bone diseases characterised by abnormal bone metabolism or resorbtion include osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

Cardiovascular diseases include hypertension or myocardiac ischemia; atherosclerosis; functional or organic venous insufficiency; varicose therapy; haemorrhoids; and shock states associated with a marked drop in arterial pressure (e.g. septic shock).

Neurodegenerative diseases include dementia, particularly degenerative dementia (including senile dementia, dementia with Lewy bodies, Alzheimer's disease, Pick's disease, Huntingdon's chorea, Parkinson's disease and Creutzfeldt-Jakob disease, Amyotrophic Lateral Sclerosis (ALS) and motor neuron disease); vascular dementia (including multi-infarct dementia); as well as dementia associated with intracranial space occupying lesions; trauma; infections and related conditions (including HIV infection, meningitis and shingles); metabolism; toxins; anoxia and vitamin deficiency; and mild cognitive impairment associated with ageing, particularly Age Associated Memory Impairment.

The compounds of formula (I) or pharmaceutically acceptable salts may also be useful for neuroprotection and in the treatment of neurodegeneration following trauma such as stroke, cardiac arrest, pulmonary bypass, traumatic brain injury, spinal cord injury or the like.

The compounds or pharmaceutically acceptable salts of the present invention may also be useful in the treatment of malignant cell growth and/or metastasis, and myoblastic leukaemia.

Complications of Type 1 diabetes include diabetic microangiopathy, diabetic retinopathy, diabetic nephropathy, macular degeneration, glaucoma, nephrotic syndrome, aplastic anaemia, uveitis, Kawasaki disease and sarcoidosis.

Kidney dysfunction includes nephritis, glomerulonephritis, particularly mesangial proliferative glomerulonephritis and nephritic syndrome.

It is to be understood that reference to treatment includes both treatment of established symptoms and prophylactic treatment, unless explicitly stated otherwise.

According to a further aspect of the invention, we therefore provide a compound of formula (I) or a pharmaceutically acceptable salt thereof for use in therapy and/or for use in human or veterinary medicine.

According to another aspect of the invention, we provide a compound of formula (I) or a pharmaceutically acceptable salt thereof for use in the treatment or prevention (e.g. treatment) of a condition which is mediated by P2X7 receptors, for example a condition or disease disclosed herein (in particular pain, inflammation or a neurodegenerative disease, more particularly pain such as inflammatory pain, neuropathic pain or visceral pain), e.g. in a mammal such as a human or rodent e.g. human or rat e.g. human.

According to a further aspect of the invention, we provide a method of treating a human or animal (e.g. rodent e.g. rat) subject, for example a human subject, suffering from a condition which is mediated by P2X7 receptors, for example a condition or disease disclosed herein (in particular pain, inflammation or a neurodegenerative disease, more particularly pain such as inflammatory pain, neuropathic pain or visceral pain), which comprises administering to said subject an effective amount of a compound of formula (I) or a pharmaceutically acceptable salt thereof.

According to a further aspect of the invention we provide a method of treating a human or animal (e.g. rodent e.g. rat) subject, for example a human subject, suffering from pain, inflammation, an immunological disease, a bone disease or a neurodegenerative disease (in particular pain, inflammation or a neurodegenerative disease, more particularly pain such as inflammatory pain, neuropathic pain or visceral pain), which method comprises administering to said subject an effective amount of a compound of formula (I) or a pharmaceutically acceptable salt thereof.

According to a yet further aspect of the invention we provide a method of treating a human or animal (e.g. rodent e.g. rat) subject, for example a human subject, suffering from inflammatory pain, neuropathic pain or visceral pain which method comprises administering to said subject an effective amount of a compound of formula (I) or a pharmaceutically acceptable salt thereof.

According to a further aspect of the invention we provide a method of treating a subject, for example a human subject, suffering from Alzheimer's disease which method comprises administering to said subject an effective amount of a compound of formula (I) or a pharmaceutically acceptable salt thereof.

According to another aspect of the invention, we provide the use of a compound of formula (I) or a pharmaceutically acceptable salt thereof for the manufacture of a medicament for the treatment or prevention (e.g. treatment) of a condition which is mediated by the action of P2X7 receptors, for example a condition or disease disclosed herein (in particular pain, inflammation or a neurodegenerative disease, more particularly pain such as inflammatory pain, neuropathic pain or visceral pain), e.g. in a mammal such as a human or rodent e.g. human or rat e.g. human.

According to another aspect of the invention we provide the use of a compound of formula (I) or a pharmaceutically acceptable salt thereof for the manufacture of a medicament for the treatment or prevention (e.g. treatment) of pain, inflammation, an immunological disease, a bone disease or a neurodegenerative disease (in particular pain, inflammation or a neurodegenerative disease, more particularly pain such as inflammatory pain, neuropathic pain or visceral pain), e.g. in a mammal such as a human or rodent e.g. human or rat e.g. human.

According to another aspect of the invention we provide the use of a compound of formula (I) or a pharmaceutically acceptable salt thereof for the manufacture of a medicament for the treatment or prevention (e.g. treatment) of inflammatory pain, neuropathic pain or visceral pain, e.g. in a mammal such as a human or rodent e.g. human or rat e.g. human.

In one aspect of the invention we provide the use of a compound of formula (I) or a pharmaceutically acceptable salt thereof for the manufacture of a medicament for the treatment or prevention (e.g. treatment) of Alzheimer's disease, e.g. in a mammal such as a human or rodent e.g. human or rat e.g. human.

In order to use a compound of formula (I) or a pharmaceutically acceptable salt thereof for the treatment of humans and other mammals it is normally formulated in accordance with standard pharmaceutical practice as a pharmaceutical composition. Therefore in another aspect of the invention there is provided a pharmaceutical composition comprising a compound of formula (I), or a pharmaceutically acceptable salt thereof, adapted for use in human or veterinary medicine.

In order to use a compound of formula (I) or a pharmaceutically acceptable salt thereof in therapy, it will normally be formulated into a pharmaceutical composition in accordance with standard pharmaceutical practice. The present invention also provides a pharmaceutical composition, which comprises a compound of formula (I), or a pharmaceutically acceptable salt thereof, and optionally a pharmaceutically acceptable carrier or excipient.

The pharmaceutical composition may be for use in a method of treatment or in a use or in a treatment or prevention, as described herein.

A pharmaceutical composition of the invention, which may be prepared by admixture, for example at ambient temperature and atmospheric pressure, is usually adapted for oral, parenteral or rectal administration. As such, the pharmaceutical composition may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain excipients, such as binding agents, fillers, tabletting lubricants, disintegrants and/or acceptable wetting agents. The tablets may be coated, e.g. according to methods known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are for example prepared utilising a compound of the invention or pharmaceutically acceptable salt thereof and a sterile vehicle. In one particular embodiment, the compound or salt, depending on the vehicle and concentration used, is either suspended or dissolved in the vehicle. In preparing solutions, the compound or salt can be dissolved for injection and filter sterilised before filling into a suitable vial or ampoule and sealing. In one embodiment, adjuvant(s) such as a local anaesthetic, a preservative and/or a buffering agent is/are dissolved in the vehicle. To enhance the stability, the composition can for example be frozen after filling into the vial and the water removed under vacuum. Parenteral suspensions are typically prepared in substantially the same manner, except that the compound or salt is typically suspended in the vehicle instead of being dissolved, and sterilization is not usually readily accomplished by filtration. The compound or salt can be sterilised e.g. by exposure to ethylene oxide before suspension in a sterile vehicle. In a particular embodiment, a surfactant or wetting agent is included in the composition to facilitate uniform distribution of the compound or salt of the invention.

In one embodiment, the composition contains from 0.1% to 99% by weight, in particular from 10 to 60% by weight, of the active material (the compound or pharmaceutically acceptable salt of the invention), e.g. depending on the method of administration.

The dose of the compound or pharmaceutically acceptable salt thereof used in the treatment or prevention (e.g. treatment) of the aforementioned disorders/diseases/conditions may vary in the usual way with the seriousness of the disorders, the weight of the sufferer, and/or other similar factors. However, as a general guide, in one embodiment a suitable unit dose of 0.05 to 1000 mg, for example 0.05 to 200 mg, such as 20 to 40 mg, of the compound or pharmaceutically acceptable salt of the invention (measured as the compound), may be used.; In one embodiment, such a unit dose is for administration once a day e.g. to a mammal such as a human; alternatively such a unit dose may be for administration more than once (e.g. twice) a day e.g. to a mammal such as a human. Such therapy may extend for a number of weeks or months.

Combinations

Compounds of formula (I) or salts thereof may be used in combination with other therapeutic agents, for example medicaments which are or may be useful in the treatment of the above mentioned disorders.

Suitable examples of other such therapeutic agents may include a β2-agonist (also known as β2 adrenoceptor agonists; e.g. formoterol) and/or a corticosteroid (e.g. budesonide, fluticasone (e.g. as propionate or furoate esters), mometasone (e.g. as furoate), beclomethasone (e.g. as 17-propionate or 17,21-dipropionate esters), ciclesonide, triamcinolone (e.g. as acetonide), flunisolide, rofleponide and butixocort (e.g. as propionate ester), for the treatment of respiratory disorders (such as asthma and chronic obstructive pulmonary disease (COPD)) as described in WO 2007/008155 and WO 2007/008157.

A further therapeutic agent may include a 3-hydroxy-3-methylglutaryl coenzyme A (HMG CoA) reductase inhibitor (e.g. atorvastatin, fluvastatin, lovastatin, pravastatin, rosuvastatin, and simvastatin) for the treatment of cardiovascular disorders (such as atherosclerosis) as described in WO 2006/083214.

A further therapeutic agent may include a non-steroid anti-inflammatory drug (NSAID; e.g. ibuprofen, naproxen, aspirin, celecoxib, diclofenac, etodolac, fenoprofen, indomethacin, ketoprofen, ketoralac, oxaprozin, nabumetone, sulindac, tolmetin, rofecoxib, valdecoxib, lumaricoxib, meloxicam, etoricoxiband and parecoxib) for the treatment of an inflammatory disease or disorder (such as rheumatoid arthritis or osteoarthritis) as described in WO 2005/025571.

A further therapeutic agent may include a tumour necrosis factor α (TNFα) inhibitor (e.g. Etanercept or an anti-TNFα antibody such as Infliximab and Adalimumab) for the treatment of an inflammatory disease or disorder (such as rheumatoid arthritis or osteoarthritis) as described in WO 2004/105798.

A further therapeutic agent may include 2-hydroxy-5-[[4-[(2-pyridinylamino)sulfonyl]phenyl]azo]benzoic acid (sulfasalazine) for the treatment of an inflammatory disease or disorder (such as rheumatoid arthritis) as described in WO 2004/105797.

A further therapeutic agent may include N-[4-[[(2,4-diamino-6-pteridinyl)methyl]methylamino]benzoyl]-L-glutamic acid (methotrexate) for the treatment of an inflammatory disease or disorder (such as rheumatoid arthritis) as described in WO 2004/105796.

A further therapeutic agent may include an inhibitor of pro TNFα convertase enzyme (TACE) for the treatment of an inflammatory disease or disorder (such as rheumatoid arthritis) as described in WO 2004/073704.

A further therapeutic agent may include:

a) sulfasalazine;

b) a statin, such as atorvastatin, lovastatin, pravastatin, simvastatin, fluvastatin, cerivastatin, crilvastatin, dalvastatin, rosuvastatin, tenivastatin, fluindostatin, velostatin, dalvastatin, nisvastatin, bervastatin, pitavastatin, rivastatin, glenvastatin, eptastatin, tenivastatin, flurastatin, rosuvastatin or itavastatin;

c) a glucocorticoid agent, such as dexamethasone, methylprednisolone, prednisolone, prednisone and hydrocortisone;

d) an inhibitor of p38 kinase;

e) an anti-IL-6-receptor antibody;

f) anakinra;

g) an anti-IL-1 monoclonal antibody;

h) an inhibitor of JAK3 protein tyrosine kinase;

i) an anti-macrophage colony stimulation factor (M-CSF) monoclonal antibody; or

j) an anti-CD20 monoclonal antibody, such as rituximab, PRO70769, HuMax-CD20 (Genmab AJS), AME-133 (Applied Molecular Evolution), or hA20 (Immunomedics, Inc.)

for the treatment of an IL-1 mediated disease (such as rheumatoid arthritis) as described in WO 2006/003517.

When the compounds are used in combination with other therapeutic agents, the compounds may be administered either sequentially or simultaneously by any convenient route.

The invention thus provides, in a further aspect, a combination comprising a compound of formula (I) or a pharmaceutically acceptable salt thereof together with a further therapeutic agent or agents, e.g. as described herein.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable salt thereof is used in combination with a second therapeutic agent active against the same disease state the dose of each compound may differ from that when the compound is used alone.

The following Descriptions and Examples illustrate the preparation of compounds of the invention but are not intended to be limiting.

### EXAMPLES

The general methods (a)-(c) for the preparation of compounds of the present invention, along with the synthetic methods outlined in Schemes 1-3 above, are further illustrated by the following examples.

Room temperature can for example be about 15-30° C., such as about 20-25° C.

### Example 1

### N-[(2-chloro-4-fluorophenyl)methyl]-2-(1,4-dimethyl-1H-pyrazol-5-yl)acetamide (E1)

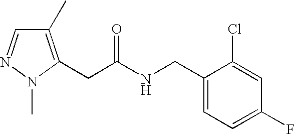

A mixture of (1,4-dimethyl-1H-pyrazol-5-yl)acetic acid and (1,4-dimethyl-1H-pyrazol-3-yl)acetic acid (1.2 g, 2. mmol, prepared as described below), 1-hydroxybenzotriazole hydrate (324 mg, 2.4 mmol), 1-ethyl-3-(3-dimethylaminopropyl)carbodiimide hydrochloride (460 mg, 2.4 mmol) and N-ethyl morpholine (690 mg, 0.76 ml, 6 mmol) in dichloromethane (10 ml) was stirred at room temperature for 10 minutes. A solution of [(2-chloro-4-difluorophenyl)methyl]amine (319 mg, 2 mmol) in dichloromethane (1 ml) was added and the reaction stirred at room temperature for 4 hours. The reaction mixture was diluted with saturated sodium bicarbonate solution and dichloromethane. The organic layer was separated washed with water and brine, dried and evaporated. The residue was purified by column chromatography on silica gel eluting with ethyl acetate/hexane mixtures (1:1 to neat ethyl acetate), followed by silica gel chromatography eluting with dichloromethane/methanol (40:1) to give N-[(2-chloro-4-fluorophenyl)methyl]-2-(1,4-dimethyl-1H-pyrazol-5-yl)acetamide.

LC/MS [M+H]⁺=329, retention time=2.35 minutes.

The mixture of (1,4-dimethyl-1H-pyrazol-5-yl)acetic acid and (1,4-dimethyl-1H-pyrazol-3-yl)acetic acid used in the above procedure was prepared as follows:

(i) A stirred suspension of sodium hydride (60% dispersion in mineral oil, 1.2 g, 30 mmol) in dry tetrahydrofuran (100 ml) under argon was treated with N,N,N′N′-tetramethyl-1,2-ethanediamine (3.36 g, 4.4 ml, 29 mmol). The resulting mixture was cooled to −20° C. (internal T) and methyl 3-oxopentanoate 3.25 g, 3.2 ml, 25 mmol) was added dropwise over 5 minutes. Reaction was stirred at −20° C. for 15 minutes. n-Butyl lithium (2.5 M in hexanes, 19.2 ml, 48 mmol) was added dropwise over 15 minutes. The yellow solution was stirred at −20° C. for 15 minutes. Ethyl formate (2.37 g, 2.6 ml, 32 mmol) was added over 1 minute; temperature rose to −5° C. and was re-cooled to −20° C. Reaction was stirred at −20° C. for 15 minutes, allowed to warm to room temperature and stirred for 15 minutes and then re-cooled to −15° C. Reaction was quenched with 2N hydrochloric acid solution (70 ml). The reaction was warmed to room temperature and then diluted with diethyl ether. The organic layer was separated and washed with water (×2). The aqueous layers were combined and saturated with sodium chloride, and the mixture was extracted with ethyl acetate (×3). The ether layer was washed with saturated sodium bicarbonate solution and water. The combined aqueous layers were acidified with 2N hydrochloric acid solution and the solution was extracted into ethyl acetate (×3). The ethyl acetate extracts were combined, dried over magnesium sulfate and solvent evaporated to afford methyl 5-hydroxy-4-methyl-3-oxo-4-pentenoate (LC/MS [M+Na]⁺=181, retention time=1.37 minutes) which was used in the next step.

(ii) A solution of methyl 5-hydroxy-4-methyl-3-oxo-4-pentenoate (1.98 g, 12.5 mmol) in tetrahydrofuran (20 ml) was treated with a solution of methyl hydrazine (632 mg, 0.73 ml, 13.75 mmol) in tetrahydrofuran (10 ml). The reaction was stirred at room temperature for 30 minutes. The reaction mixture was diluted with acetone and water and the solution was stirred at room temperature for 15 minutes. The organic solvent was evaporated and the aqueous solution was diluted with ethyl acetate and water. The organic layer was washed with brine, dried and evaporated. The residue was purified by column chromatography on silica gel, eluting with methanol in dichloromethane (1:50 to 1:40) to give a mixture of methyl(1,4-dimethyl-1H-pyrazol-5-yl)acetate and methyl(1,4-dimethyl-1H-pyrazol-3-yl)acetate (LC/MS [M+H]⁺=169, retention time=1.70 minutes.) which was used in the next step.

(iii) A solution of the mixture of methyl(1,4-dimethyl-1H-pyrazol-5-yl)acetate and methyl(1,4-dimethyl-1H-pyrazol-3-yl)acetate (1.35 g, 8 mmol) in methanol (12 ml) was treated with 2N sodium hydroxide solution (12 ml) and the mixture was heated at reflux for 3 hours. The reaction mixture was cooled to room temperature and the methanol was evaporated. The pH of the aqueous solution was adjusted to pH 3 with 5M hydrochloric acid solution. The solvent was evaporated and the residue dried to afford a mixture (1,4-dimethyl-1H-pyrazol-5-yl)acetic acid and (1,4-dimethyl-1H-pyrazol-3-yl)acetic acid (LC/MS [M+H]⁺=155, retention time=1.28 and 1.35 minutes) which was used in subsequent reactions without further purification.

### Example 2

### N-{[2-chloro-3-(trifluoromethyl)phenyl]methyl}-2-(1,4-dimethyl-1H-pyrazol-5-yl)acetamide (E2)

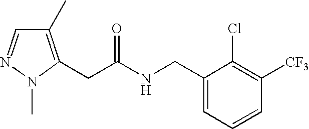

The title compound was prepared in a manner analogous to that described for Example 1 above substituting {[2-chloro-3-(trifluoromethyl)phenyl]methyl}amine for the [(2-chloro-4-fluorophenyl)methyl]amine used in the above procedure. LC/MS [M+H]⁺=346 retention time=2.55 minutes.

### Example 3

### N-[(2-chloro-3,4-difluorophenyl)methyl]-2-[4-methyl-1-(2,2,2-trifluoroethyl)-1H-pyrazol-5-yl]acetamide (E3)

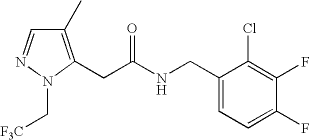

To a suspension of a mixture of [4-methyl-1-(2,2,2-trifluoroethyl)-1H-pyrazol-5-yl]acetic acid and [4-methyl-1-(2,2,2-trifluoroethyl)-1H-pyrazol-3-yl]acetic acid (3.5 mmol) in dichloromethane (15 ml) was added 1-hydroxybenzotriazole hydrate (566 mg, 4.2 mmol), 1-ethyl-3-(3-dimethylaminopropyl)carbodiimide hydrochloride (805 mg, 4.2 mmol) and N-ethyl morpholine (1.2 g, 1.3 ml, 10.5 mmol), and the reaction stirred at room temperature for 30 minutes. [(2-Chloro-3,4-difluorophenyl)methyl]amine hydrochloride (899 mg, 4.2 mmol) was added and the reaction stirred at room temperature for a further 24 hours. Dichloromethane and saturated sodium bicarbonate solution were added and the product extracted into dichloromethane (×4). Combined organic extracts were washed with water (×2), brine (×1) and dried over magnesium sulfate. Solvent was evaporated in vacuo. Crude product was purified by column chromatography on silica gel eluting with 0-50% ethyl acetate in hexane, and then 50%-100% ethyl acetate in hexane. Latter fractions were combined and solvent evaporated in vacuo to give crude product which was purified further by mass directed auto prep. Relevant fractions were combined, solvent evaporated in vacuo and solid triturated with diethyl ether. The solid was then re-crystallised in 1:1 hexane:dichloromethane, and triturated with methanol to afford N-[(2-chloro-3,4-difluorophenyl)methyl]-2-[4-methyl-1-(2,2,2-trifluoroethyl)-1H-pyrazol-5-yl]acetamide.

LC/MS [M+H]⁺=382, retention time=2.78 minutes.

The mixture of [4-methyl-1-(2,2,2-trifluoroethyl)-1H-pyrazol-5-yl]acetic acid and [4-methyl-1-(2,2,2-trifluoroethyl)-1H-pyrazol-3-yl]acetic acid (3.5 mmol) used in the above procedure was prepared as follows:

(i) Methyl 5-hydroxy-4-methyl-3-oxo-4-pentenoate was prepared as described above in Example 1. To methyl 5-hydroxy-4-methyl-3-oxo-4-pentenoate (1.3 g, 8.1 mmol) in tetrahydrofuran (15 ml) was added 2,2,2-trifluoroethylhydrazine (70% solution in water, 1.0 g, 1.1 ml, 8.9 mmol) in tetrahydrofuran (7 ml). The reaction was stirred at room temperature for 2 hours. A further 0.6 eq of 2,2,2-trifluoroethylhydrazine (70% solution in water, 0.6 ml, 4.7 mmol) was added and the reaction stirred at room temperature for a further 2 hours. 10 m of acetone and 10 ml of water were added and the reaction stirred at room temperature for a further 20 minutes. Organic solvent was evaporated in vacuo, and the aqueous residue diluted with ethyl acetate and brine solution. Product was extracted into ethyl acetate (×3) and the combined organic extracts were dried over magnesium sulfate and solvent evaporated in vacuo. Crude product was purified further by column chromatography on silica gel, eluting with 0-4% methanol in dichloromethane to give a mixture of methyl[4-methyl-1-(2,2,2-trifluoroethyl)-1H-pyrazol-5-yl]acetate and methyl[4-methyl-1-(2,2,2-trifluoroethyl)-1H-pyrazol-3-yl]acetate which was used in the next step.

(ii) To a mixture of methyl[4-methyl-1-(2,2,2-trifluoroethyl)-1H-pyrazol-5-yl]acetate and methyl[4-methyl-1-(2,2,2-trifluoroethyl)-1H-pyrazol-3-yl]acetate (820 mg, 3.5 mmol) in methanol (6 ml) was added 2N sodium hydroxide solution (6 ml), and the resulting solution heated at reflux for 3 hours. Reaction was allowed to cool to room temperature and then organic solvent evaporated in vacuo. Aqueous residue was washed with dichloromethane (×1) and then acidified by the addition of 2N hydrochloric acid solution. Product was extracted into dichloromethane (×5) and the combined organic extracts evaporated in vacuo. Aqueous layer was evaporated in vacuo, combined with organic residue to give a crude mixture of [4-methyl-1-(2,2,2-trifluoroethyl)-1H-pyrazol-5-yl]acetic acid and [4-methyl-1-(2,2,2-trifluoroethyl)-1H-pyrazol-3-yl]acetic acid (LC/MS [M+]⁺=223, retention time=1.68 minutes.) which was used in the subsequent reaction.

The [(2-chloro-3,4-difluorophenyl)methyl]amine hydrochloride required for the synthesis of N-[(2-chloro-3,4-difluorophenyl)methyl]-2-[4-methyl-1-(2,2,2-trifluoroethyl)-1H-pyrazol-5-yl]acetamide (E3) was prepared in the following manner:

(i) A solution of N,N,N′,N′-tetramethylethylenediamine (39.6 ml, 264 mmol) in tetrahydrofuran (170 ml) was cooled under argon to −70° C. before the addition of sec-butyl lithium (205 ml, 288 mmol). To the mixture 3,4-difluorobenzoic acid (19 g, 120 mmol) was then added as a solution in tetrahydrofuran (80 ml) over a period of 40 minutes ensuring that the temperature of the mixture did not rise above −60° C. The mixture was then stirred at a temperature of −68° C. to −70° C. for 1 hr before adding a solution of hexachloroethane (100 g, 422 mmol) in tetrahydrofuran (170 ml) over a period of 35 minutes whilst keeping the temperature of the mixture below −60° C. The mixture was stirred at a temperature of −65° C. to −70° C. for 2 hrs. The mixture was allowed to warm to −10° C. and then water (500 ml) was added to quench the reaction. The mixture was diluted with diethyl ether (250 ml) and the two resulting layers were separated. The aqueous layer was acidified to pH1 using concentrated aqueous hydrogen chloride and then extracted with 2×500 ml aliquots of diethyl ether. The combined organic extracts were passed through a hydrophobic frit and reduced in vacuo to give a yellow solid. This was recrystallised from ethyl acetate to give two crops (8.35 g and 4.47 g) of 2-chloro-3,4-difluorobenzoic acid.

(ii) 2-Chloro-3,4-difluorobenzoic acid (2 g, 10.4 mmol) was treated with thionyl chloride (3.04 ml) and the mixture was heated to 80° C. for 90 minutes. The mixture was then cooled and reduced in vacuo. The residue was dissolved in anhydrous 1,4-dioxane (10 ml) and the mixture was then cooled in an ice-water bath. 0.88 Ammonia (aqueous, 25 ml) was added dropwise to the mixture which was subsequently allowed to warm to 22° C. over a period of 2 hrs. This process was repeated using 10.8 g of 2-chloro-3,4-difluorobenzoic acid, 8.2 ml of thionyl chloride, and 45 ml of 0.88 ammonia and then both mixtures were combined and partitioned between ethyl acetate (150 ml) and water (100 ml). The aqueous layer was separated and extracted with 2×150 ml aliquots of ethyl acetate. The combined organic extracts were then washed with saturated aqueous sodium hydrogen carbonate solution (100 ml), dried using a hydrophobic frit, and reduced in vacuo to give 2-chloro-3,4-difluorobenzamide (11.86 g) as a white solid.

LC/MS [M+H]⁺=192/194, retention time=1.69 minutes.

(iii) 2-Chloro-3,4-difluorobenzamide (11.85 g, 62 mmol) was dissolved in tetrahydrofuran (200 ml) and treated with 1 M borane tetrahydrofuran (247 ml, 247 mmol). The mixture was heated to 70° C. and stirred for 18 hrs. The mixture was then cooled in an ice-water bath and concentrated aqueous hydrogen chloride (150 ml) was added dropwise. Heating, with stirring, at 70° C. was then resumed for a further 2 hrs. The mixture was then allowed to cool and the solvent was evaporated in vacuo. The residue was partitioned between ethyl acetate (200 ml) and 2N aqueous hydrogen chloride (200 ml). The aqueous layer was separated and the pH was adjusted to 8-9 by dropwise addition of 5N aqueous sodium hydroxide solution. The resulting cloudy suspension was extracted with ethyl acetate (4×200 ml) and the combined organic extracts were then passed through a hydrophobic frit and reduced in volume to ∫200 ml. The mixture was then acidified by the addition of 1M ethereal hydrogen chloride (100 ml) resulting in formation of a precipitate. The solvent was evaporated in vacuo to give a white solid. The solid was recrystallised from methylated spirit (60 ml) to give three crops of [(2-chloro-3,4-difluorophenyl)methyl]amine hydrochloride (combined mass=4.46 g) as a white solid.

### Example 4

### N-[(2-chloro-3,4-difluorophenyl)methyl]-2-(1,4-dimethyl-1H-pyrazol-5-yl)acetamide (E4)

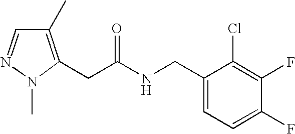

A mixture of (1,4-dimethyl-1H-pyrazol-5-yl)acetic acid and (1,4-dimethyl-1H-pyrazol-3-yl)acetic acid (462 mg, 3.0 mmol, prepared as described below) in dichloromethane (20 ml) was treated with 1-hydroxybenzotriazole hydrate (486 mg, 3.6 mmol), 1-ethyl-3-(3-dimethylaminopropyl)carbodiimide hydrochloride (691 mg, 3.6 mmol) and N-ethyl morpholine (1.04 g, 1.2 ml, 9.0 mmol). The mixture was stirred at room temperature for 10 minutes and a solution of [(2-Chloro-3,4-difluorophenyl)methyl]amine hydrochloride (639 mg, 3.6 mmol) (e.g. prepared as described in Example 3) in dichloromethane (10 ml) was added and the reaction was stirred at room temperature for 2 hours. The reaction mixture was diluted with dichloromethane and the solution was washed with saturated sodium bicarbonate solution, water, citric acid solution, water and brine. The organic solution was dried and the solvent was evaporated. The residue was purified by column chromatography on silica gel eluting with dichloromethane:methanol (30:1). The resulting solid was triturated with diethyl ether, collected and dried to afford N-[(2-chloro-3,4-difluorophenyl)methyl]-2-(1,4-dimethyl-1H-pyrazol-5-yl)acetamide.

LC/MS [M+H]⁺=314, retention time=2.33 minutes.

The mixture of (1,4-dimethyl-1H-pyrazol-5-yl)acetic acid and (1,4-dimethyl-1H-pyrazol-3-yl)acetic acid used in the above procedure was prepared as follows:

(i) Methyl hydrazine (690 mg, 0.8 ml, 15 mmol) was added in one portion to a solution of methyl 5-hydroxy-4-methyl-3-oxo-4-pentenoate (1.58 g, 10 mmol) in water (5 ml). The methyl 5-hydroxy-4-methyl-3-oxo-4-pentenoate was prepared as described above in Example 1. The reaction mixture was stirred for 15 minutes. Acetone (10 ml) was added and the mixture was stirred at room temperature for 1 hour. The organic solvent was evaporated and the aqueous mixture was partitioned between dichloromethane and saturated sodium bicarbonate solution. The organic layer was separated, washed with brine, dried and evaporated to give a 6:1 mixture of methyl(1,4-dimethyl-1H-pyrazol-5-yl)acetate and methyl(1,4-dimethyl-1H-pyrazol-3-yl)acetate which was used in the next step.

(ii) A solution of the mixture of methyl(1,4-dimethyl-1H-pyrazol-5-yl)acetate and methyl(1,4-dimethyl-1H-pyrazol-3-yl)acetate (1.38 g, 8.2 mmol) in methanol (12 ml) was treated with 2N sodium hydroxide solution (12.3 ml, 24.6 mmol) and the solution was heated at reflux overnight. Reaction was cooled to room temperature and the methanol was evaporated. The residue was acidified with 5M hydrochloric acid solution to pH2. The mixture was extracted with ethyl acetate (×3) and then with chloroform (×2). The organic extracted were combined, washed with brine, dried and evaporated. The residue was triturated with ether/hexane (1:1) and the solid collected, washed with hexane and dried to afford a 6:1 mixture (1,4-dimethyl-1H-pyrazol-5-yl)acetic acid and (1,4-dimethyl-1H-pyrazol-3-yl)acetic acid which was used in subsequent reactions without further purification.

### Example 5

### N-{[2-chloro-3-(trifluoromethyl)phenyl]methyl}-2-(1-methyl-1H-pyrazol-5-yl)acetamide (E5)

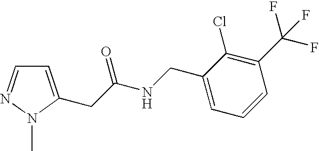

A suspension of (1-methyl-1H-pyrazol-5-yl)acetic acid (6 mmol, prepared as described below), 1-hydroxybenzotriazole hydrate (972 mg, 7.2 mmol), 1-ethyl-3-(3-dimethylaminopropyl)carbodiimide hydrochloride (1.38 g, 7.2 mmol) and N-ethyl morpholine (2.76 g, 3 ml, 24 mmol) in dichloromethane (50 ml) was stirred at room temperature for 10 minutes. A solution of {[2-chloro-3-(trifluoromethyl)phenyl]methyl}amine (1.25 g, 6 mmol) in dichloromethane (5 ml) was added and the reaction stirred at room temperature for 1 hour. The reaction mixture was diluted with dichloromethane and the organic solution was washed with saturated sodium bicarbonate solution, water, citric acid, water and brine, dried and evaporated. The residue was triturated with ether/hexane (1:1) to give N-{[2-chloro-3-(trifluoromethyl)phenyl]methyl}-2-(1-methyl-1H-pyrazol-5-yl)acetamide (E5).

LC/MS [M+H]⁺=332, retention time=2.45 minutes.

The (1-methyl-1H-pyrazol-5-yl)acetic acid used in the above procedure was prepared as follows:

(i) 1-Methyl-1H-pyrazole-5-carbaldehyde was prepared as follows (c.f. Bioorg. Med. Chem., 2001, 9, 961-982): A solution of 1-methyl-1H-pyrazole (11.0 g, 0.134 mol) in tetrahydrofuran (200 ml) was stirred at −70° C. under argon. n-Butyl lithium (2.5 M in hexanes, 58.8 ml, 0.147 mol) was added dropwise maintaining the internal reaction temperature below −60° C. The mixture was cooled to −70° C. and N,N-dimethylformamide (40 ml) was added dropwise. The reaction mixture was allowed to warm to room temperature overnight. The mixture was cooled to 0° C. and water (200 ml) was added followed by ethyl acetate (300 ml). The organic layer was separated and the aqueous layer was re-extracted with ethyl acetate. The combined organic extracts were washed with water, brine, then dried and evaporated. The residue was purified by flash chromatography on silica gel eluting with 0-100% ethyl acetate in hexanes to afford 1-methyl-1H-pyrazole-5-carbaldehyde which was used in the next step.

(ii) (1-Methyl-1H-pyrazol-5-yl)methanol was prepared as follows (c.f. Bioorg. Med. Chem., 2001, 9, 961-982): A stirred solution of 1-methyl-1H-pyrazole-5-carbaldehyde (5.2 g, 47 mmol) in ethanol (50 ml) at 5° C. was treated with sodium borohydride (893 mg, 23. 5mmol). The reaction mixture was stirred at 5° C. for 30 minutes, hydrochloric acid (5N, 10 ml) was added and the organic solvent was evaporated. The residue was partitioned between ethyl acetate and water. The layers were separated and the aqueous solution was saturated with sodium chloride and re-extracted with ethyl acetate. The combined organic extracts were washed with brine, then dried and evaporated to afford (1-methyl-1H-pyrazol-5-yl)methanol which was used in the next step.

(iii) A solution of (1-methyl-1H-pyrazol-5-yl)methanol (4.4 g, 40 mmol) in dichloromethane (30 ml) was treated with thionyl chloride (2.14 g, 1.3 ml, 18 mmol) and the reaction was stirred at room temperature for 3 hours. The solvent was evaporated and the residue co-evaporated with dichloromethane. The residue was partitioned between dichloromethane and water. The organic phase was separated and washed with saturated sodium bicarbonate solution, water, brine, then dried and evaporated to afford 5-(chloromethyl)-1-methyl-1H-pyrazole which was used in the next step.

(iv) A solution of 5-(chloromethyl)-1-methyl-1H-pyrazole (1.3 g, 10 mmol) in dimethylsulfoxide (15 ml) was treated with sodium cyanide (588 mg, 12 mmol) and the reaction mixture was heated at 50° C. under argon for 3 hours. The reaction mixture was cooled to room temperature, water was added and the mixture was extracted with chloroform. The organic layer was separated, washed with water, brine, dried and evaporated. The residue was purified by silica gel chromatography eluting with 50-100% ethyl acetate in hexanes to afford (1-methyl-1H-pyrazol-5-yl)acetonitrile which was used in the next step.

(v) A solution of (1-methyl-1H-pyrazol-5-yl)acetonitrile (800 mg, 6.6 mmol) in 1,4-dioxane (5 ml) and hydrochloric acid (5M, 5 ml) was heated at reflux for 20 hours. The reaction was cooled to room temperature and the solvent was evaporated. The residue was partitioned between water and chloroform. The aqueous layer was evaporated and dried over the weekend at 40° C. in the vacuum oven to afford crude 1-methyl-1H-pyrazol-5-yl)acetic acid which was used in the next step.

### Example 6

### 2-(4-chloro-1-methyl-1H-pyrazol-5-yl)-N-{[2-chloro-3-(trifluoromethyl)phenyl]methyl}acetamide (E6)

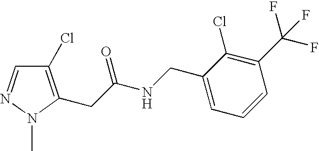

A solution of N-{[2-chloro-3-(trifluoromethyl)phenyl]methyl}-2-(1-methyl-1H-pyrazol-5-yl)acetamide (e.g. prepared as described in example 5) (166 mg, 0.5 mmol) in N,N-dimethylformamide (2 ml) was treated with N-chlorosuccinimide (73 mg, 0.55 mmol). The reaction was stirred at room temperature for 1 hour and the reaction mixture was then heated at 60° C. for 22 hours. The reaction mixture was cooled to room temperature; ice water and dichloromethane were added. The mixture was diluted with sodium thiosulfate solution and saturated sodium bicarbonate solution. The organic layer was separated, washed with water, brine, then dried and evaporated. The residue was triturated with diethyl ether and the solid collected and dried to afford 2-(4-chloro-1-methyl-1H-pyrazol-5-yl)-N-{[2-chloro-3-(trifluoromethyl)phenyl]methyl}acetamide (E6).

LC/MS [M+H]⁺=366, retention time=2.74 minutes.

### Example 7

### 2-(4-Bromo-1-methyl-1H-pyrazol-5-yl)-N-{[2-chloro-3-(trifluoromethyl)phenyl]methyl}acetamide (E7)

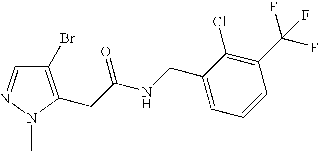

A solution of N-{[2-chloro-3-(trifluoromethyl)phenyl]methyl}-2-(1-methyl-1H-pyrazol-5-yl)acetamide (e.g. prepared as described in example 5) (166 mg, 0.5 mmol) in N,N-dimethylformamide (2 ml) was treated with N-bromosuccinimide (196 mg, 0.55 mmol). The reaction was stirred at room temperature for 1 hour. The reaction was diluted with water and chloroform, sodium thiosulfate solution and saturated sodium bicarbonate solution. The organic layer was separated, washed with water, brine, then dried and evaporated. The residue was triturated with ether and the solid collected and dried to afford 2-(4-bromo-1-methyl-1H-pyrazol-5-yl)-N-{[2-chloro-3-(trifluoromethyl)phenyl]methyl}acetamide (E7).

LC/MS [M+H]⁺=410, retention time=2.77 minutes.

### Example 8

### N-{[2-chloro-3-(trifluoromethyl)phenyl]methyl}-2-(4-fluoro-1-methyl-1H-pyrazol-5-yl)acetamide (E8)

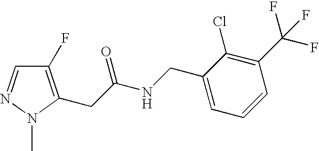

A solution of N-{[2-chloro-3-(trifluoromethyl)phenyl]methyl}-2-(1-methyl-1H-pyrazol-5-yl)acetamide (e.g. prepared as described in example 5) (166 mg, 0.5 mmol) in N,N-dimethylformamide (2 ml) was stirred with Selectfluor™ (1-(chloromethyl)-4-fluoro-1,4-diazoniabicyclo[2.2.2]octane ditetrafluoroborate, CAS[140681-55-6], e.g. available from Aldrich) (265 mg, 0.75 mmol) at room temperature for 18 hours. Additional Selectfluor™ (177 mg, 0.5 mmol) was added and stirring was continued at room temperature for 3 hours and then the reaction was heated at 50° C. for 4 hours. After cooling to room temperature the solution was diluted with water and dichloromethane. The organic layer was separated, washed with water, brine, dried and evaporated. The residue was purified by column chromatography on silica gel eluting with dichloromethane/methanol (40:1-20:1). The resulting solid was triturated with ether, collected and dried to afford N-{[2-chloro-3-(trifluoromethyl)phenyl]methyl}-2-(4-fluoro-1-methyl-1H-pyrazol-5-yl)acetamide (E8).

LC/MS [M+H]⁺=350, retention time=2.62 minutes.

### Examples 9-10

In a manner analogous to that described for Example 4 above the compounds tabulated below (Table 1) were prepared by substituting the appropriate amine (or salt thereof) for the [(2-chloro-3,4-difluorophenyl)methyl]amine hydrochloride used in the above procedure. All of the amines used in Table 1 are available from commercial sources or can be prepared e.g. using routes described previously in the chemical literature or analogous methods.

TABLE 1

| | | | Retention |
|---|---|---|---|
| | | | time |
| Example no. | Chemical name | [M + H]⁺ | (mins) |
| E9 | 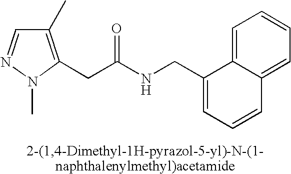 | 294 | 2.38 |
| E10 | 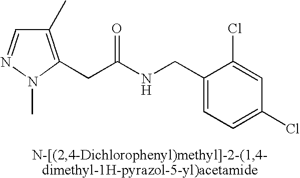 | 312 | 2.47 |

### Examples 11-13

In a manner analogous to that described for Example 5 above the compounds tabulated below (Table 2) were prepared by substituting the appropriate amine (or salt thereof) for the {[2-chloro-3-(trifluoromethyl)phenyl]methyl}amine used in the above procedure. All of the amines in Table 2 are available from commercial sources or can be prepared e.g. using routes described previously in the chemical literature or analogous methods. The [(2-Chloro-3,4-difluorophenyl)methyl]amine hydrochloride used to prepare example 12 could be prepared as described in Example 3.

TABLE 2

| | | | Retention |
|---|---|---|---|
| | | | time |
| Example no. | Chemical name | [M + H]⁺ | (mins) |
| E11 | 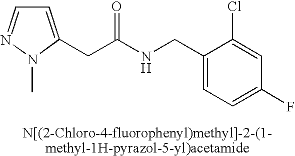 | 282 | 2.14 |
| E12 | 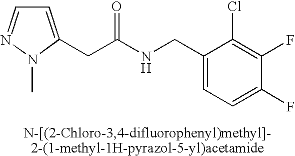 | 300 | 2.24 |
| E13 | 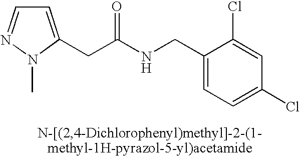 | 298 | 2.36 |

### Example 14

### N-[(2-Chloro-4-fluorophenyl)methyl]-2-(4-chloro-1-methyl-1H-pyrazol-5-yl)acetamide (E14)

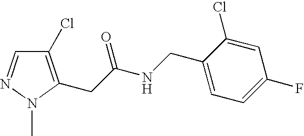

A solution of N-[(2-chloro-4-fluorophenyl)methyl]-2-(1-methyl-1H-pyrazol-5-yl)acetamide (e.g. prepared as described in example 11) (141 mg, 0.5 mmol) in N,N-dimethylformamide (2 ml) was treated with N-chlorosuccinimide (73 mg, 0.55 mmol). The reaction mixture was stirred at room temperature for 1 hour and the reaction mixture was then heated at 60° C. for 22 hours. Additional N-chlorosuccinimide (17 mg, 0.125 mmol) was added and the reaction mixture was heated at 60° C. for 6 hours. The reaction mixture was cooled to room temperature and diluted with water and dichloromethane. The mixture was diluted with sodium thiosulfate solution and saturated sodium bicarbonate solution. The organic layer was separated, washed with water, brine, dried and evaporated. The residue was purified by mass-directed automated HPLC. The resulting solid was triturated with diethyl ether and the solid collected and dried to give N-[(2-chloro-4-fluorophenyl)methyl]-2-(4-chloro-1-methyl-1H-pyrazol-5-yl)acetamide.

LC/MS [M+H]⁺=316, retention time=2.48 minutes.

### Example 15

### N-[(2-Chloro-3,4-difluorophenyl)methyl]-2-(4-chloro-1-methyl-1H-pyrazol-5-yl)acetamide (E15)

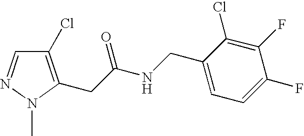

A solution of N-[(2-chloro-3,4-difluorophenyl)methyl]-2-(1-methyl-1H-pyrazol-5-yl)acetamide (e.g. prepared as described in example 12) (150 mg, 0.5 mmol) in N,N-dimethylformamide (2 ml) was treated with N-chlorosuccinimide (73 mg, 0.55 mmol). The reaction mixture was stirred at room temperature for 1 hour and the reaction mixture was then heated at 60° C. for 22 hours. Additional N-chlorosuccinimide (17 mg, 0.125 mmol) was added and the reaction mixture was heated at 60° C. for 6 hours. The reaction mixture was cooled to room temperature and diluted with water and dichloromethane. The mixture was diluted with sodium thiosulfate solution and saturated sodium bicarbonate solution. The organic layer was separated, washed with water, brine, dried and evaporated. The residue was purified by mass-directed automated HPLC. The resulting solid was triturated with diethyl ether and the solid collected and dried to give N-[(2-chloro-3,4-difluorophenyl)methyl]-2-(4-chloro-1-methyl-1H-pyrazol-5-yl)acetamide.

LC/MS [M+H]⁺=334, retention time=2.55 minutes.

### Example 16

### 2-(4-Chloro-1-methyl-1H-pyrazol-5-yl)-N-[(2,4-dichlorophenyl)methyl]acetamide (E16)

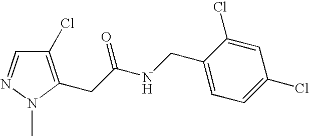

A solution of N-[(2,4-dichlorophenyl)methyl]-2-(1-methyl-1H-pyrazol-5-yl)acetamide (e.g. prepared as described in example 13) (149 mg, 0.5 mmol) in N,N-dimethylformamide (2 ml) was treated with N-chlorosuccinimide (73 mg, 0.55 mmol). The reaction mixture was stirred at room temperature for 1 hour and the reaction mixture was then heated at 60° C. for 22 hours. The reaction mixture was cooled to room temperature and diluted with water and dichloromethane. The mixture was diluted with sodium thiosulfate solution and saturated sodium bicarbonate solution. The organic layer was separated, washed with water, brine, dried and evaporated. The residue was triturated with diethyl ether and the solid collected and dried to give 2-(4-chloro-1-methyl-1H-pyrazol-5-yl)-N-[(2,4-dichlorophenyl)methyl]acetamide.

LC/MS [M+H]⁺=332, retention time=2.68 minutes.

### Example 17

### N-[(2-Chloro-4-fluorophenyl)methyl]-2-(4-fluoro-1-methyl-1H-pyrazol-5-yl)acetamide (E17)

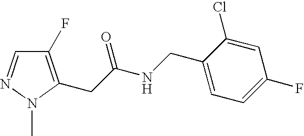

A solution of N-[(2-chloro-4-fluorophenyl)methyl]-2-(1-methyl-1H-pyrazol-5-yl)acetamide (e.g. prepared as described in example 11) (282 mg, 1 mmol) in N,N-dimethylformamide (4 ml) was treated with Selectfluor™ (Aldrich) (708 mg, 2 mmol) and the reaction mixture was heated at 50° C. for 5 hours. After cooling to room temperature the solution was diluted with water and dichloromethane. The organic layer was separated, washed with water, brine, then dried and evaporated. The residue was purified by mass-directed automated HLPC to give N-[(2-chloro-4-fluorophenyl)methyl]-2-(4-fluoro-1-methyl-1H-pyrazol-5-yl)acetamide. LC/MS [M+H]⁺=300, retention time=2.32 minutes.

### Example 18

### N-[(2-Chloro-3,4-difluorophenyl)methyl]-2-(4-fluoro-1-methyl-1H-pyrazol-5-yl)acetamide (E18)

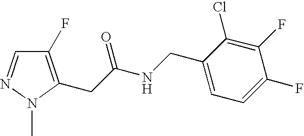

A solution of N-[(2-chloro-3,4-difluorophenyl)methyl]-2-(1-methyl-1H-pyrazol-5-yl)acetamide (e.g. prepared as described in example 12) (299.5 mg, 1 mmol) in N,N-dimethylformamide (4 ml) was treated with Selectfluor™ (Aldrich) (708 mg, 2 mmol) and the reaction mixture was heated at 50° C. for 5 hours. After cooling to room temperature the solution was diluted with water and dichloromethane. The organic layer was separated, washed with water, brine, dried and evaporated. The residue was purified by mass-directed automated HLPC to give N-[(2-chloro-3,4-difluorophenyl)methyl]-2-(4-fluoro-1-methyl-1H-pyrazol-5-yl)acetamide. LC/MS [M+H]⁺=318, retention time=2.41 minutes.

### Example 19

### N-[(2,4-Dichlorophenyl)methyl]-2-(4-fluoro-1-methyl-1H-pyrazol-5-yl)acetamide (E19)

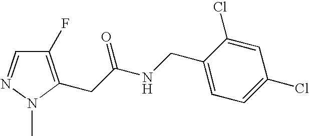

A solution of N-[(2,4-dichlorophenyl)methyl]-2-(1-methyl-1H-pyrazol-5-yl)acetamide (e.g. prepared as described in example 13) (282 mg, 1 mmol) in N,N-dimethylformamide (4 ml) was treated with Selectfluor™ (Aldrich) (708 mg, 2 mmol) and the reaction mixture was heated at 50° C. for 5 hours. After cooling to room temperature the solution was diluted with water and dichloromethane. The organic layer was separated, washed with water, brine, dried and evaporated. The residue was purified by mass-directed automated HLPC to give N-[(2,4-dichlorophenyl)methyl]-2-(4-fluoro-1-methyl-1H-pyrazol-5-yl)acetamide. LC/MS [M+H]⁺=316, retention time=2.53 minutes.

### Example 20

### N-[(2,4-Dichlorophenyl)methyl]-2-(4-iodo-1-methyl-1H-pyrazol-5-yl)acetamide (E20)

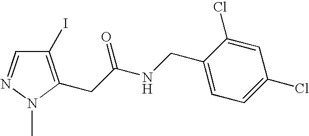

A solution of N-[(2,4-dichlorophenyl)methyl]-2-(1-methyl-1H-pyrazol-5-yl)acetamide (e.g. prepared as described in example 13) (298 mg, 1 mmol) in N,N-dimethylformamide (4 ml) was treated with N-iodosuccinimide (248 mg, 1.1 mmol). The reaction mixture was stirred at room temperature for 18 hours. Additional N-iodosuccinimide (113 mg, 0.5 mmol) was added and the reaction mixture was heated at 50° C. for 2 hours. The reaction mixture was diluted with dichloromethane and the solution was washed with sodium thiosulfate solution, saturated sodium bicarbonate solution, water and brine, dried and evaporated. The residue was triturated with ether and the solid collected and dried to afford N-[(2,4-dichlorophenyl)methyl]-2-(4-iodo-1-methyl-1H-pyrazol-5-yl)acetamide. LC/MS [M+H]⁺=424, retention time=2.75 minutes.

Liquid Chromatography/Mass Spectrometry

Analysis of the above Examples by Liquid Chromatography/Mass Spectrometry (LC/MS) was carried out using the following apparatus and conditions:

Hardware

Agilent 1100 Gradient Pump

Agilent 1100 Autosampler

Agilent 1100 DAD Detector

Agilent 1100 Degasser

Agilent 1100 Oven

Agilent 1100 Controller

Waters ZQ Mass Spectrometer

Sedere Sedex 85

Software

Waters MassLynx version 4.0 SP2

Column

The column used is a Waters Atlantis, the dimensions of which are 4.6 mm×50 mm.

The stationary phase particle size is 3 μm.

Solvents

A: Aqueous solvent=Water+0.05% Formic Acid

B: Organic solvent=Acetonitrile+0.05% Formic Acid

Method

The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

The above method has a flow rate of 3ml/mins.

The injection volume for the generic method is 5 ul.

The column temperature is 30 deg.

The UV detection range is from 220 to 330 nm.

Pharmacological Data

Compounds of the invention may be tested for in vitro biological activity at the P2X7 receptor in accordance with the following studies:

Ethidium Accumulation Assay

Studies were performed using NaCl assay buffer of the following composition (in mM): 140 mM NaCl, HEPES 10, N-methyl-D-glucamine 5, KCl 5.6, D-glucose 10, CaCl₂ 0.5 (pH 7.4). HEK293 cells, expressing human recombinant P2X7 receptors, were grown in poly-L-lysine pretreated 96 well plates for 18-24 h. (The cloning of the human P2X7 receptor is described in U.S. Pat. No. 6,133,434). The cells were washed twice with 350 μl of assay buffer before addition of 50 μl of antagonist. The cells were then incubated at room temperature (19-21° C.) for 30 min before addition of ATP and ethidium (100 μM final assay concentration). The ATP concentration was chosen to be close to the EC₈₀ for the receptor type and was 1 mM for studies on the human P2X7 receptor. Incubations were continued for 8 or 16 min and were terminated by addition of 25 μl of 1.3M sucrose containing 5 mM of the P2X7 receptor antagonist reactive black 5 (Aldrich). Cellular accumulation of ethidium was determined by measuring fluorescence (excitation wavelength of 530nm and emission wavelength of 620 nm) from below the plate with a Canberra Packard Fluorocount (Pangbourne, UK). Antagonist pIC₅₀ values for blocking ATP responses were determined using iterative curve fitting techniques.

Fluorescent Imaging Plate Reader (FLIPR) Ca Assay

Studies were performed using NaCl assay buffer of the following composition (in mM) for human P2X7: 137 NaCl; 20 HEPES; 5.37 KCl; 4.17 NaHCO₃; 1 CaCl₂; 0.5 MgSO₄; and 1 g/L of D-glucose (pH 7.4).

HEK293 cells, expressing human recombinant P2X7 receptors, were grown in poly-L-lysine pretreated 384 well plates for 42-48 h. (The cloning of the human P2X7 receptor is described in U.S. Pat. No. 6,133,434). The cells were washed three times with 80 μl of assay buffer, loaded for 1 h at 37° C. with 2 μM Fluo4 (Teflabs), washed three times again, and left with 30 μl buffer before the addition of 10 μl of 4× concentrated antagonist. The cells were then incubated at room temperature for 30 mins before addition (online, by FLIPR384 or FLIPR3 instrument (Molecular Devices)) of Benzoylbenzoyl-ATP (BzATP) 60 μM final assay concentration. The BzATP concentration was chosen to be close to the EC₈₀ for the receptor type. Incubations and reading were continued for 90 sec, and intracellular calcium increase was determined by measuring fluorescence (excitation wavelength of 488 nm and emission wavelength of 516 nm) from below the plate, with FLIPR CCD camera. Antagonist pIC₅₀ values for blocking BzATP responses were determined using iterative curve fitting techniques.

The compounds of Examples 1 to 20 were tested in the FLIPR Ca Assay for human P2X7 receptor antagonist activity and found to have pIC50 values>4.7 in the FLIPR Ca Assay. The compounds of Examples 1 to 8 and Examples 10 to 20 were tested in the Ethidium Accumulation Assay for human P2X7 receptor antagonist activity and were found to have pIC50 values>6.5 in the Ethidium Accumulation Assay; the compounds of Examples 2 to 8, 10, 14, 15, 16, 18, 19 and 20 were found to have pIC50 values of about 7.0 or more in the Ethidium Accumulation Assay.

1-18. (canceled)

19. A compound of formula (I): 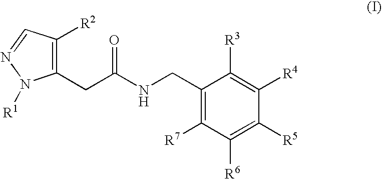
wherein:
R¹ represents C₁₋₆ alkyl or C₃₋₆ cycloalkyl, either of which is optionally substituted with 1, 2 or 3 halogen atoms;
R² represents hydrogen, halogen, C₁₋₆ alkyl or C₃₋₆ cycloalkyl; and either of said C₁₋₆ alkyl or C₃₋₆ cycloalkyl is optionally substituted with 1, 2 or 3 halogen atoms; and
R³, R⁴, R⁵, R⁶ and R⁷ independently represent hydrogen, halogen, cyano, C₁₋₆ alkyl, C₂₋₆ alkenyl, C₂₋₆ alkynyl, C₃₋₆ cycloalkyl or phenyl, and any of said C₁₋₆ alkyl, C₂₋₆ alkenyl, C₂₋₆ alkynyl, C₃₋₆ cycloalkyl or phenyl is optionally substituted with 1, 2 or 3 halogen atoms;
or R⁶ and R⁷ together with the carbon atoms to which they are attached form a benzene ring which is optionally substituted with 1, 2 or 3 halogen atoms;
with the proviso that when R³ and R⁷ independently represent hydrogen or fluorine, at least one of R⁴, R⁵ and R⁶ is a halogen atom, or only one of R⁴, R⁵ and R⁶ is a CF₃ group;
or a pharmaceutically acceptable salt thereof.

20. The compound or salt according to claim 19, wherein R¹ represents C₁₋₆ alkyl optionally substituted with 1, 2 or 3 halogen atoms.

21. The compound or salt according to claim 19, wherein R¹ represents methyl or trifluoroethyl.

22. The compound or salt according to claim 19, wherein R² represents hydrogen, unsubstituted C₁₋₆ alkyl or halogen.

23. The compound or salt according to claim 19, wherein R² represents hydrogen, methyl or halogen.

24. The compound or salt according to claim 19, wherein R² represents methyl or halogen.

25. The compound or salt according to claim 19, wherein R³, R⁴, R⁵, R⁶ and R⁷ independently represent hydrogen, halogen, cyano, trifluoromethyl or unsubstituted C₁₋₆ alkyl.

26. The compound or salt according to claim 19, wherein R³, R⁴, R⁵, R⁶ and R⁷ independently represent hydrogen, chlorine, fluorine, bromine, methyl or trifluoromethyl.

27. The compound or salt according to claim 19, wherein, when R³ and R⁷ independently represent hydrogen or fluorine, at least one of R⁴, R⁵ and R⁶ is a halogen atom.

28. The compound or salt according to claim 19, wherein:
R¹ represents C₁₋₆ alkyl optionally substituted with 1, 2 or 3 halogen atoms;
R² represents hydrogen, methyl or halogen; and
R³, R⁴, R⁵, R⁶ and R⁷ independently represent hydrogen, chlorine, fluorine, bromine, methyl or trifluoromethyl.

29. The compound or salt according to claim 19, wherein:
R³ represents chlorine, R⁴ represents trifluoromethyl, and R⁵, R⁶ and R⁷ each represent hydrogen;
R³represents chlorine, R⁵ represents fluorine, and R⁴, R⁶ and R⁷ each represent hydrogen;
R³ represents chlorine, R⁴ and R⁵ both represent fluorine, and R⁶ and R⁷ both represent hydrogen; or
R³ and R⁵ both represent chlorine, and R⁴, R⁶ and R⁷ each represent hydrogen.

30. A compound which is:
N-[(2-chloro-4-fluorophenyl)methyl]-2-(1,4-dimethyl-1H-pyrazol-5-yl)acetamide;
N-{[2-chloro-3-(trifluoromethyl)phenyl]methyl}-2-(1,4-dimethyl-1H-pyrazol-5-yl)acetamide;
N-[(2-chloro-3,4-difluorophenyl)methyl]-2-[4-methyl-1-(2,2,2-trifluoroethyl)-1H-pyrazol-5-yl]acetamide;
N-[(2-chloro-3,4-difluorophenyl)methyl]-2-(1,4-dimethyl-1H-pyrazol-5-yl)acetamide;
N-{[2-chloro-3-(trifluoromethyl)phenyl]methyl}-2-(1-methyl-1H-pyrazol-5-yl)acetamide;
2-(4-chloro-1-methyl-1H-pyrazol-5-yl)-N-[2-chloro-3-(trifluoromethyl)phenyl]methyl}acetamide;
2-(4-Bromo-1-methyl-1H-pyrazol-5-yl)-N-{[2-chloro-3-(trifluoromethyl)phenyl]methyl}acetamide; or
N-{[2-chloro-3-(trifluoromethyl)phenyl]methyl}-2-(4-fluoro-1-methyl-1H-pyrazol-5-yl)acetamide;
or a pharmaceutically acceptable salt thereof.

31. A compound which is:
2-(1,4-dimethyl-1H-pyrazol-5-yl)-N-(1-naphthalenylmethyl)acetamide;
N-[(2,4-dichlorophenyl)methyl]-2-(1,4-dimethyl-1H-pyrazol-5-yl)acetamide;
N-[(2-chloro-4-fluorophenyl)methyl]-2-(1-methyl-1H-pyrazol-5-yl)acetamide;
N-[(2-chloro-3,4-difluorophenyl)methyl]-2-(1-methyl-1H-pyrazol-5-yl)acetamide;
N-[(2,4-dichlorophenyl)methyl]-2-(1-methyl-1H-pyrazol-5-yl)acetamide;
N-[(2-Chloro-4-fluorophenyl)methyl]-2-(4-chloro-1-methyl-1H-pyrazol-5-yl)acetamide;
N-[(2-Chloro-3,4-difluorophenyl)methyl]-2-(4-chloro-1-methyl-1H-pyrazol-5-yl)acetamide;
2-(4-Chloro-1-methyl-1H-pyrazol-5-yl)-N-[(2,4-dichlorophenyl)methyl]acetamide;
N-[(2-Chloro-4-fluorophenyl)methyl]-2-(4-fluoro-1-methyl-1H-pyrazol-5-yl)acetamide;
N-[(2-Chloro-3,4-difluorophenyl)methyl]-2-(4-fluoro-1-methyl-1H-pyrazol-5-yl)acetamide;
N-[(2,4-dichlorophenyl)methyl]-2-(4-fluoro-1-methyl-1H-pyrazol-5-yl)acetamide;
or
N-[(2,4-dichlorophenyl)methyl]-2-(4-iodo-1-methyl-1H-pyrazol-5-yl)acetamide;
or a pharmaceutically acceptable salt thereof.

32. A pharmaceutical composition which comprises a compound of formula (I) or a pharmaceutically acceptable salt thereof as defined in claim 19 and a pharmaceutically acceptable carrier or excipient.

33. A method of treating a human suffering from pain, inflammation or a neurodegenerative disease, which method comprises administering to said human an effective amount of the compound of formula (I) as defined in claim 19.

34. A method of treating a human suffering from inflammatory pain, neuropathic pain or visceral pain, which method comprises administering to said human an effective amount of the compound of formula (I) as defined in claim 19.
